(19) United States
(12) Patent Application Publication
(10) Pub. No.: US 2024/0287496 A1
(43) Pub. Date: Aug. 29, 2024

(54) AUTOMATED PLASMID EXTRACTION

(71) Applicant: XPRESS BIOLOGICS, Herstal (BE)

(72) Inventors: Marc DAUKANDT, Bilstain (BE); Philippe LEDENT, Liège (BE); Christian RODRIGUEZ, Spa (BE)

(21) Appl. No.: 18/572,642

(22) PCT Filed: Jul. 4, 2022
(86) PCT No.: PCT/EP2022/068461

§ 371 Date: Dec. 20, 2023

(30) Foreign Application Priority Data
Jul. 2, 2021 (BE) BE2021/5517

(51) Int. Cl. C12N 15/10

(52) U.S. Cl. CPC C12N 15/1003; C12N 15/1017

(57) ABSTRACT

A method is for discontinuous plasmid extraction from a microorganism and for purifying a plasmid of interest. The method includes adding a neutralizing solution and then a precipitation solution via a plurality of orifices, under mild mechanical stirring.

### CROSS-REFERENCE TO RELATED APPLICATIONS

The present application is a National Stage Filing of PCT International Application No. PCT/EP2022/068461 filed on Jul. 4, 2022, which claims priority to Belgian application No. BE2021/5517, filed with the Belgian Patent Office on Jul. 2, 2021; which applications are incorporated herein by reference in their entirety.

### TECHNICAL FIELD

This invention relates to the automated extraction of a plasmid of interest produced by a bacterium.

### PRIOR ART

The production of a plasmid of interest in a bacterium, such as Escherichia coli, is known, as is alkaline lysis followed by a neutralisation and precipitation step (e.g. Birnboim and Dolly, 1979, Nucleic Acids Research, 7, pages 1513-1523).

This method is simple to put into practice and works well in batch mode; it is suitable for producing small quantities of plasmids, such as in the order of grams, at most.

Patent application WO 2010/136503 (also published, among others under EP2435569B1, U.S. Pat. Nos. 8,822,672 and 9,416,400) describes a continuous plasmid extraction method, based on a piping arrangement system for the different solutions and mixtures thereof and flow control via pumps. In addition to the neutralising solution, typically composed of an acetic acid/acetate buffer, this method provides for the addition of a concentrated precipitating solution. The neutralising and precipitating solutions are mixed homogeneously by adapting the internal diameter of the piping, so as to locally create a Venturi effect. Indeed, the inventor of this patent application noted that mechanic stirring was not possible for such volumes of viscous solutions, with the risk of shearing the genomic DNA and even the plasmids, which is unacceptable, unlike mixing via the Venturi effect. In addition and advantageously, this is a single-use system, so it does not require cleaning. However, though this system is very effective for very large quantities of plasmids to be purified, for example 100 g of plasmid, it generates fixed costs (single-use device, plasmid loss) which become significant if there are smaller quantities of plasmids to be purified.

There is therefore a range of quantities which is difficult to treat using a batch method, and which cannot be very efficiently treated using the continuous method summarised above, even though the two approaches work well in their respective applications.

Indeed, the efficiency of systems in batch mode is limited by physical parameters such as the size of the containers, the time taken to bring the solutions into contact, or even the need for rapid homogenisation. Thus, in batch mode, an ideal purification is achieved using low volumes, which allows mixtures to be well controlled, but significantly complicates production on a larger scale.

### SUMMARY OF THE INVENTION

This invention relates to a method for extracting a plasmid synthesised by a microorganism comprising the successive steps of:
obtaining a receptacle 1 coupled to an extraction pump 8, said receptacle 1 being provided with mechanical stirring means 9,
obtaining a cell suspension 2 of said microorganism comprising said plasmid to be extracted;
adding to said cell suspension 2 an alkaline lysis solution 3 at a predetermined flow rate (Q1) so as to form a homogeneous mixture;
placing said homogeneous mixture in said receptacle 1 at a predetermined flow rate (Q2);
after a predetermined period, preferably with gentle stirring, adding to it a neutralising solution 5 comprising acetic acid in said receptacle at a predetermined flow rate (Q3), via a plurality of orifices 7;
after a predetermined period, adding to it a precipitating solution 6 at a predetermined flow rate (Q4) in said receptacle, via a plurality of orifices 7;
after a predetermined period with gentle stirring, extracting the suspension thus formed via said extraction pump 8 at a predetermined flow rate (Q5), then clarifying, preferably centrifuging, said extracted mixture so as to recover the clarified supernatant, comprising said plasmid of interest.

This extracted and purified plasmid can advantageously be used directly, or after sterilising filtration, ultrafiltration and/or polishing chromatography.

### BRIEF DESCRIPTION OF THE DRAWINGS

FIG. 1 shows a semi-schematic view of a container according to the invention.

FIG. 2 shows an HPLC analysis of the plasmid extracted by the automated method according to the invention.

FIG. 3 shows the effect of variations #1; #2; #3 and #4 made to the preferred automated method.

### DETAILED DESCRIPTION OF THE INVENTION

The inventors have succeeded in developing a plasmid extraction method which retains the flexibility of a batch mode method, while allowing large quantities of plasmid to be treated and with a very high extraction yield and purity level.

A first aspect of this invention is a method for extracting a synthesised plasmid comprising the successive steps of:
obtaining a receptacle 1 coupled to an extraction pump 8, said receptacle 1 being provided with mechanical stirring means 9,
obtaining a cell suspension 2 comprising said plasmid to be extracted;
adding to said cell suspension 2 an alkaline lysis solution 3 at a predetermined flow rate Q1 so as to form a homogeneous mixture;
placing said homogeneous mixture in said receptacle 1 at a predetermined flow rate Q2;
after a predetermined time, preferably with gentle stirring, adding to it at a predetermined flow rate Q3 a neutralising solution 5 comprising acetic acid in said receptacle, via a plurality of orifices 7;
after a predetermined period, adding to it a precipitating solution 6 at a predetermined flow rate Q4 in said receptacle, via a plurality of orifices 7;
after a predetermined period with gentle stirring, extracting the suspension thus formed via said extraction pump 8 at a predetermined flow rate Q5, then clarifying, preferably centrifuging, said extracted mixture so as to recover the clarified supernatant, comprising said plasmid of interest.

The cells synthesising the plasmid of interest are typically Gram-negative bacteria, for example Escherichia coli. However, other Gram-negative bacteria, or even Gram-positive bacteria, or even other microorganisms, such as Saccharomyces Sp. or Pichia Sp. may also be suitable.

The plasmid is advantageously in so-called “super coiled” form.

The size of the plasmid is not a limiting factor because this method also works with large plasmids. However, as will be described later, plasmids of larger sizes (e.g. >10 kb, for example between 10 and 20 kb, including plasmids encoding viral vectors and/or comprising repeated and/or inverted sequences) require more precise timing control, in particular with regard to the contact time with the alkaline lysis medium and the addition and contact times with the neutralising solution.

The size of the receptacle 1 is not particularly limited. Receptacles 1 that are too small do not allow the processing of sufficient quantities of plasmids, while receptacles 1 filled with excessively large quantities of solution risk requiring too much pumping time, which is disadvantageous, since this complicates or even prevents the control of different times. Indeed, since the solutions are pumped one by one, there is heterogeneity at the start of each step: pumping for too long will therefore cause double heterogeneity in terms of compositions and contact times.

The inventors found that receptacles 1 filled at the end (after incorporation of solutions 2+3+5+6) with 5 to 10 litres of solutions were very simple to use. On the other hand, receptacles 1 filled at the end (after incorporation of solutions 2+3+5+6) with 100 litres become too difficult to control. Thus, a preferred size of the usable volumes of receptacles is between 1 litres and 50 litres, preferably between 2.5 litres and 20 litres, even more preferably between 5 litres and 10 litres.

Obviously, receptacles 1 with a larger capacity, or even significantly larger (than 50 or 100 litres) can be used, even if they are only intended to be filled with a maximum of 2.5 to 10 litres.

Surprisingly, the inventors found that this heterogeneity, potentially this double heterogeneity, is not fatal, as long as it is controlled and/or is not excessive.

For example, the inventors determined that the contact time between the cell suspension and the lysis solution is advantageously between 2 and 5 minutes. Thus, the inventors deduced that solutions 2 and 3 can be added in, for example, one minute, without causing any problems.

Furthermore, preferably, the suspension of cells 2 and the lysis solution 3 are homogenised upstream of the receptacle 1, for example via a static mixing system 4.

In the context of this invention, the term “static mixer” is preferably understood to mean any device which causes turbulence to the combined flow of the suspension of cells 2 and the lysis solution 3, resulting in rapid homogenisation of this combined flow. The inventors noted that this type of mixture is advantageous in that it is not associated with excessively high shear forces.

The suspension of the cells is, preferably, a cell culture pellet which has been taken up in a TRIS-EDTA buffer; the suspension is at a concentration of 10 to 300, preferably 50 to 200, even more preferably 75 to 150, such as approximately 100 g/L (weight of the cells: total volume of the suspension).

This allows initial homogenisation without the involvement of shear forces. Since the two solutions are combined at this level, any potential heterogeneity is greatly reduced.

Preferably, the lysis solution 3 has a pH of between 12.0 and 12.5, the pH of the lysis solution is preferably fixed by an alkaline hydroxide.

This allows rapid lysis and denaturation of the genomic DNA. The use of an alkaline hydroxide, such as NaOH, allows for a very basic pH with a low buffering capacity, which will simplify subsequent neutralisation. However, the pH must be carefully fixed otherwise there is a risk of not allowing for sufficient lysis and denaturation, or, on the contrary, of irreversibly denaturing the plasmid of interest.

Preferably, the lysis solution 3 further comprises a detergent, preferably 0.1% by weight of sodium dodecyl sulfate.

Advantageously, the neutralising solution 5 has a pH of between 4.5 and 5.7; this solution preferably comprises acetic acid at a concentration greater than 1 M, even more preferably, the neutralising solution has a pH fixed at approximately 5.5 by means of an acetic acid/acetate mixture. This ensures that the neutralised solution (solutions 2, 3 and 5 combined) has a pH of lower than 7.0, preferably lower than 6.0 and, preferably (logically) a pH higher than 4.5, preferably higher than 5.0.

Preferably, the suspension comprising the plasmid remains in the presence of the neutralising solution for at least 1 minute, for example between 2 and 3 minutes. Excessive contact times allow the genomic DNA to renature, which is detrimental. Thus, in this batch process it is preferable for the neutralising 5 and precipitating 6 solutions to be added quickly.

Thus, for plasmids less than 10 kb in size, a contact time with the neutralising solution between 20 and 80 seconds is advantageous.

Conversely, for plasmids larger than 10 kb, especially for those which contain repeated and/or inverted sequences, a contact time with the neutralising solution of 1 to 3 minutes is preferred. In this case, the time for adding solutions 5 and 6 becomes a parameter that must be controlled very carefully, ideally 30 seconds each, at most.

However, the flow rates cannot be adopted with total freedom. There are physical flow constraints, therefore pressure constraints, to be respected, especially since (i) the mixture containing solutions 2+3+5, as well as (ii) solution 6 are very viscous. In addition, it is preferable to use robust and/or commonly used pumps, allowing for efficient maintenance. If necessary, in particular for large plasmids, the volumes of the different solutions will be reduced, so as to ensure that the solutions, in particular solutions 5 and 6, can be added quickly (see the above paragraph).

Peristaltic pumps are advantageous because flow rates are well controlled and they do not cause shearing.

Advantageously, the precipitating solution 6 comprises a water-soluble calcium salt (such as CaCl₂)) at a concentration of between 3.5 and 6 M, preferably approximately 5M.

Thanks to this concentrated calcium solution, the calcium concentration in the (suspension) solution after adding the precipitating solution (solutions 2, 3, 5 and 6 combined) is at least 0.8 M, for example at least 1.0 M, or even at least 1.2 M. The inventors prefer to use a very concentrated precipitating solution, despite its viscosity, so as to maintain reasonable volumes, while guaranteeing a sufficient final calcium concentration. Such a final calcium concentration (>0.8 M, or even >1M) allows for the precipitation of impurities, in particular of RNA and genomic DNA which has not had time to denature, but also of proteins and endotoxins (if the starting cell synthesises them), or at least a significant part of the endotoxins.

Advantageously, the plurality of orifices 7 is a perforated piping system. This system can be a simple perforated pipe, a coil, or even a ring.

The advantage of the plurality of orifices is to inject the neutralising solution 5 and/or the precipitating solution 6 into a plurality of locations in the receptacle, which results in a plurality of microheterogeneities, rather than mass heterogeneity. This, combined with gentle mechanical stirring 9, allows for non-shearing and rapid homogenisation. This responds to the double problem of heterogeneity mentioned above.

The inventors noted to their surprise that this approach allowed for sufficiently rapid homogenisation of the solutions, without causing the above-mentioned shearing issues.

Advantageously, the plurality of orifices 7 used for the addition of the neutralising solution 5 and the plurality of orifices 7 used for the addition of the precipitating solution 6 are the same plurality of orifices 7: the containers containing the precipitating and neutralising solutions being connected upstream of receptacle 1. This makes it possible to avoid having to have multiple devices, and also makes it possible to purge all of the neutralising solution.

Advantageously, the neutralising 5 and/or precipitating solutions 6 are added substantially via the bottom of the receptacle 1, preferably at least 30, 40, or even (at least or exactly) 50% by weight and/or volume of said neutralising and/or precipitating solutions is added to the bottom 20% of said receptacle. This allows controlled injection of solutions and increases the diffusion thereof.

According to a variant, the neutralising solution 5 is added substantially via the bottom of the receptacle 1, preferably at least 30, 40, or even (at least or exactly) 50% by weight and/or volume of the neutralising solution 5 is added to the bottom 20% of said receptacle and the precipitating solution 6 is added substantially at the top of the receptacle 1, preferably at least 30, 40, or even (at least or exactly) 50% by weight of the precipitating solution 6 is added to the top 20% of said receptacle. This can be done by varying the height of the system comprising the plurality of orifices 7, or by using two systems comprising the plurality of separate orifices 7.

According to a second variant, the neutralising solution 5 is added substantially to the top of the receptacle 1, preferably at least 30, 40, or even (at least or exactly) 50% by weight of the neutralising solution 5 are added to the top 20% of said receptacle and the precipitating solution 6 is added substantially via the bottom of the receptacle 1, preferably at least 30, 40, or even (at least or exactly) 50% by weight and/or volume of the precipitating solution 6 is added to the bottom 20% of said receptacle. This can be done by varying the height of the system comprising the plurality of orifices 7, or by using two systems comprising the plurality of separate orifices 7. Preferably the flow rates Q1, Q2, Q3, Q4 and Q5 are independently between 0.5 L/min and 25 L/min, more preferably between 1 L/min and 10 L/min.

The flow rates Q1, Q2, Q3, Q4 and/or Q5 can be, independently, constant; or the flow rates Q1, Q2, Q3, Q4 and/or Q5 can be, independently, variable. For example, the flow rates Q1, Q2, Q3, Q4 and/or Q5, preferably Q3 and/or Q4, can be increased over time (lowest when pumping starts and highest when it finishes) so as to limit heterogeneities when adding these viscous solutions. An advantageous way of increasing flow rates is to maintain a constant, or substantially constant, flow/volume ratio. Independently of the flow rates Q1, Q2, Q3 and/or Q4 which may be variable (increasing over time), the flow rate Q5 is preferably constant. Extraction 8 is preferably very rapid, so as to prevent aggregates from disturbing it. Thus, the flow rate Q5 is preferably constant and the fastest. The flow rates Q1 and Q2 are preferably matched (determined together) so (i) all of the cell suspension 2 and the lysis solution 3 are pumped at the same time and (ii) so the concentration of the mixture (content in cells, pH), for example at the outlet of the static mixer 4, is constant.

Advantageously, gentle stirring 9 generates insufficient shear stresses to shear the plasmid DNA or the genomic DNA from the host.

Thus, a possible preliminary step consists of testing the acceptability of the shear stresses, depending on the type of microorganism, the size of the plasmid to be purified and the chosen concentrations of the solutions.

A related aspect of this invention is a method for purifying the plasmid of interest from the clarified supernatant according to the above, comprising the steps of:
harvesting of the clarified supernatant comprising said plasmid,
filtration of the clarified supernatant on a filter with a porosity of between 0.1 and 0.4 μm, preferably between 0.15 and 0.3 μm, favourably around 0.2 μm, to obtain a filtered solution comprising said plasmid,
optionally, ultrafiltration of the filtered solution,
chromatography polishing on an anion exchange resin, preferably said polishing comprising a step of washing the plasmid fixed on the resin by means of a solution comprising Polyoxyethylene (10) isooctylcyclohexyl ether;
formulation of the plasmid into a final solution.

### EXAMPLES

It is understood that this invention is in no way limited to the embodiments described above and that many modifications can be made thereto without departing from the scope of the attached claims.

### COMPARATIVE EXAMPLE

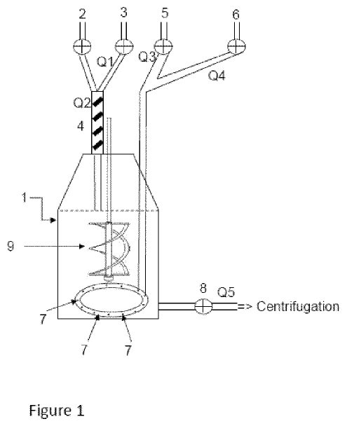

The inventors sought to develop an “in-bottle” batch method. For ease of reference, references will be made to FIG. 1, which could not have elements 4 and 7, as well as the piping and pump system upstream of container 1. To do this, 0.3 litres of a concentrated cell suspension 2 (100 g of cells/litre of Tris-EDTA medium) producing a plasmid was inserted into container 1 with a capacity of 2 litres, then 0.3 litres of alkaline lysis solution 3 (NaOH; pH 12.5; SDS 0.1% by weight) were added quickly and container 1 was stirred manually by the operator for exactly 90 seconds. Then, 0.6 litres of neutralising solution 5 (AcOH 15% v:v/3M potassium acetate, pH between 4.5 and 5.7) was added quickly and the contents of container 1 were stirred manually for exactly 90 seconds. Finally, 0.23 litres of precipitating solution 6 (CaCl₂), 5M) was added quickly and the contents of container 1 were manually stirred for exactly 90 seconds; then the mixture was extracted for clarification by centrifugation.

The inventors only recovered 200 mg of the plasmid per litre, and this plasmid contained measurable quantities of genomic DNA and RNA, which requires, in practice, adding a chromatography step, which leads to additional costs and a loss of yield. By repeating this manual method with the aim of improving it, the inventors obtained yields which varied from single to double, but also variable contents of contaminating RNA and plasmid in “Open circular” form, the best yield being associated with an increased content of contaminants. Thus, the ratio of plasmid DNA in open circular form varied from 7 to 12%; values above 10% are considered high. Indeed, the desired form is the so-called “super coiled” form, and it is difficult to separate these two forms by HPLC or by any other means, as the two cells tend to overlap.

The inventors, faced with these results, believe that the increase in contaminant levels is due to a more significant application of shear forces during this manual extraction, and that the intensity of these shear forces varies depending on the batch (identity of the operator, the operator's potential tiredness level).

### Example 1

The inventors had the idea to develop this system which they considered to be ineffective. To do this, 1.2 litres of a concentrated cell suspension 2 (100 g of cells/litre in the same Tris-EDTA medium) containing the plasmid was inserted into the container 1 with a capacity of 10 litres, via a peristaltic pump at the same time as 1.2 litres of the alkaline lysis solution 3 (NaOH; pH 12.5; SDS 0.1% by weight), with these two solutions passing through a static mixer 4. The injection time was 30 seconds. The container underwent slow mechanical stirring 9 (non-shearing) for exactly 90 seconds. Then, 2.4 L of the same neutralising solution 5 as in the comparative example was added quickly (flow rate of 6 L/min) at the bottom, via a peristaltic pump and a diffusion ring perforated with numerous orifices 7, and the contents of container 1 were mechanically stirred 9, but in a non-shearing manner, for exactly 90 seconds. Finally, 0.92 L of the precipitating solution 6 (CaCl₂), 5M) was added quickly (flow rate of 2.3 L/min) via a peristaltic pump and the same device perforated with orifices 7, still under slow mechanical stirring 9 for exactly 90 seconds; then the mixture was extracted 8 for clarification by centrifugation.

The inventors recovered approximately 400 mg of plasmid/L, and this plasmid did not contain measurable quantities of genomic DNA and little RNA.

Since stirring is carried out for 90 seconds, the inventors conclude that this device can be easily adapted (volumes, flow rates) by slightly modifying the duration of stirring.

### Example 2

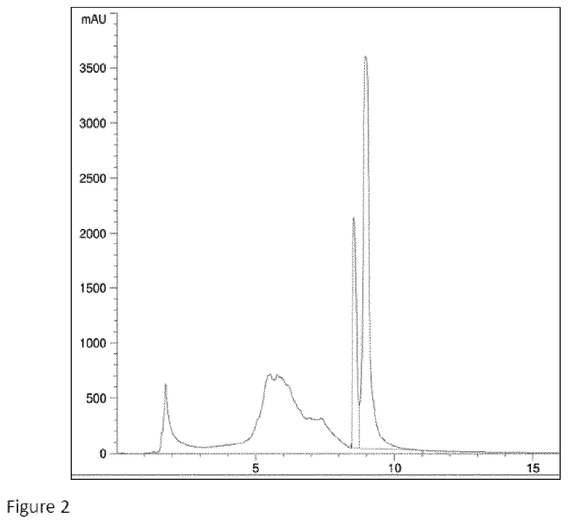

The inventors then analysed the plasmid obtained by the method according to the invention by HPLC. This allowed for an extraction yield of 84%, and an “open circular” plasmid content of only 7.2% (therefore almost 93% in the super coiled form), which is the preferred form. Via HPLC, also, the RNA contents are very low: see FIG. 2, the first peak on the left representing the residual salts, the second set of peaks representing the RNA, and the double peak representing the plasmid, with the peak on the right, the largest, being the supercoiled form, for which, here, the HPLC signal shows is saturated.

In addition to this, the main advantages of the method according to the invention relate to reproducibility and the possibility of production on a larger scale: a plurality of reactors according to the invention can be managed in parallel by a single operator, whereas manual stirring requires one operator per bottle, and cannot be carried out too quickly.

### Example 3—Comparative Example

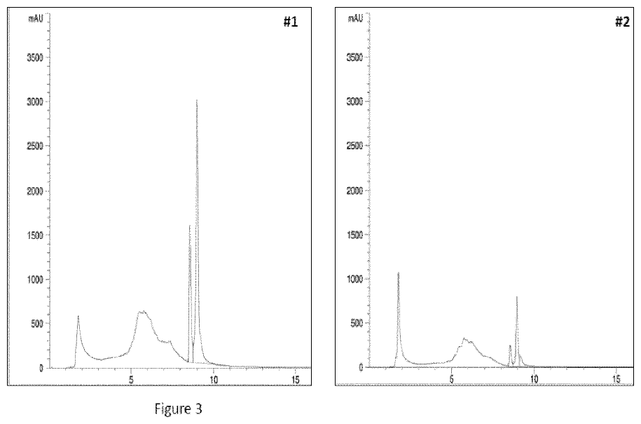

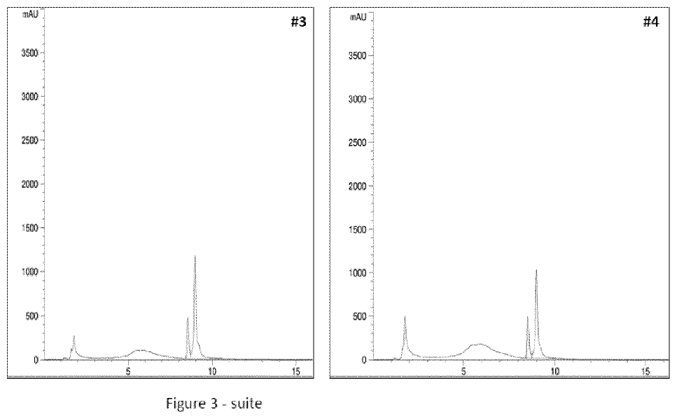

The inventors compared 4 conditions with the condition according to the invention (see the HPLC profiles in FIG. 3).
1. The diffusion ring is placed in the middle of the bottle, not at the bottom;
2. The diffusion ring is omitted;
3. The static mixer is omitted;
4. Both the diffusion ring and the static mixer are omitted.

A visual inspection shows strong heterogeneities in the upper part of the bottles for conditions #2 and #3. Condition #4 seems less affected by heterogeneities, after a simple visual analysis. Condition #1 shows intermediate-level heterogeneities between the method where the diffusion ring is at the bottom, and the method where the diffusion ring is moved to the middle of the container.

The filtration parameters were also decreased, where it was necessary to change the filters twice for conditions #1 and #4 and once for condition #2.

However, the most significant differences are found in the yield, which drops to 67% for condition #1, but only 22.6, 14 and 30% for conditions #2, #3 and #4. The proportion of “open circular” plasmids also increases, to 16% for condition #1, 9.9% for condition #3 and 13.7% for condition #4. RNA contamination (RNA: plasmid) also increases, except for in condition #3.

1. A method for extracting a plasmid synthesized by a microorganism comprising the successive steps of:
obtaining a receptacle coupled to an extraction pump, said receptacle comprising a mechanical stirrer,
obtaining a cell suspension of said microorganism comprising said plasmid to be extracted;
adding to said cell suspension an alkaline lysis solution at a predetermined first flow rate to form a homogeneous mixture;
placing at a predetermined second flow rate said homogeneous mixture in said receptacle;
after a predetermined period with stirring, adding to said homogeneous mixture at a predetermined third flow rate a neutralizing solution comprising acetic acid in said receptacle, via a plurality of orifices;
after a predetermined period, adding to said homogeneous mixture at a predetermined fourth flow rate a precipitating solution in said receptacle, via a plurality of the orifices;
after the predetermined period with stirring, extracting the suspension formed via said extraction pump at a predetermined fifth flow rate, then clarifying said extracted mixture to recover a clarified supernatant, comprising said plasmid of interest.

2. The method according to claim 1 wherein the cell suspension and the lysis solution are homogenized upstream of the receptacle via a static mixing system.

3. The method according to claim 1, wherein the lysis solution has a pH of between 12.0 and 12.5, wherein the pH is fixed by a hydroxide of an alkali.

4. The method according to claim 3, wherein the lysis solution further comprises a detergent, comprising 0.1% by weight of sodium dodecyl sulfate.

5. The method according to claim 1, wherein the neutralizing solution has a pH of between 4.5 and 5.7.

6. The method according to claim 1, wherein the neutralising neutralizing solution comprises acetic acid at a concentration greater than 1 M, wherein the neutralizing solution has a pH fixed by an acetic acid/acetate mixture.

7. The method according to claim 1, wherein the solution is neutralized to a pH of less than 7.0.

8. The method according to claim 1, wherein the precipitating solution comprises a water-soluble calcium salt at a concentration of between 3.5 and 6 M.

9. The method according to claim 8, wherein the concentration of calcium in the solution after addition of the precipitating solution is at least 0.8 M.

10. The method according to claim 1, wherein the plurality of orifices is a perforated piping system.

11. The method according to claim 1, wherein the plurality of orifices used for addition of the neutralizing solution and the plurality of orifices used for addition of the precipitating solution are the same plurality of orifices.

12. The method according to claim 1, wherein the neutralizing solution and/or the precipitating solution are added substantially at a bottom of the receptacle, at least 50% by weight of said neutralizing and/or precipitating solutions is added to the bottom 20% of said receptacle.

13. The method according to claim 1, wherein the first, second, third, fourth and fifth flow rates are independently between 0.5 L/min and 25 L/min.

14. The method according to claim 1, wherein the stirring generates shear stresses that are insufficient to shear plasmid DNA or genomic DNA from a host.

15. The method according to claim 1, wherein the solutions are added and/or extracted by one or more peristaltic pumps.

16. A process for purifying the plasmid of interest from the clarified supernatant according to claim 1, comprising the steps of:
harvesting of the clarified supernatant comprising said plasmid,
filtering the clarified supernatant on a filter with a porosity of between 0.1 and 0.4 μm, to obtain a filtered solution comprising said plasmid,
ultrafiltering the filtered solution,
chromatography polishing on an anion exchange resin, said polishing comprising a step of washing the plasmid fixed on the resin by a solution comprising Polyoxyethylene isooctylcyclohexyl ether;
formulation of formulating the plasmid into a final solution.
